(19) United States
(12) Plant Patent Application Publication
(10) Pub. No.: US 2005/0262600 P1
(43) Pub. Date: Nov. 24, 2005

(54) Miniature rose plant 'PACtwo'

(50) ROSA HYBRIDA; SUPER MINIATURE ROSE

(76) Inventor: Dan Jauchen, Santa Barbara, CA (US)

Correspondence Address: Dan Jauchen;905 South Patterson Avenue, P.O. Box 6354, Santa Barbara, CA, 93111, US

(21) Appl. No.: 10/851,297

(22) Filed: May 24, 2004

(51) Int. Cl. A01H 5/00

(52) U.S. Cl. PLT/125

(57) ABSTRACT

A new miniature rose plant which has abundant, yellow colored flowers and attractive foliage. The variety successfully propagates from softwood cuttings and is suitable for year round production in commercial glass houses as a flowering pot plant. This new and distinct variety has shown to be uniform and stable in the resulting generations from asexual propagation.

### BACKGROUND OF THE INVENTION

The present invention constitutes a new and distinct variety of miniature rose plant, which was developed by artificially pollinating an unnamed seedling(not patent in the US) with an unnamed seedling (not patent in the US). The two parents were crossed in the summer of 2001 and the resulting seed was sown in December 2001, in a controlled glasshouse environment. Out of these seedlings one seedling was selected, as the new variety and named ‘PACtwo’. The new rose may be distinguished from its seed parent, an unnamed seedling, by the following combination of characteristics:
1. The unnamed seedling has a breeding background in unnamed seedlings.
2. ‘PACtwo’ has big double flowers, while the unnamed seedling has big single flowers.
3. ‘PACtwo’ has yellow colored petals, while the unnamed seedling has white petals.

The new variety may distinguished from its pollen parent, an unnamed seedling created by the same inventor, by the following combination of characteristics:
1. The unnamed seedling has a breeding background in unnamed seedlings.
2. ‘PACtwo’ has big flowers and foliage as compared to the unnamed seedling.
3. ‘PACtwo’ has yellow colored petals, while the unnamed seedling has dark orange petals.

### BRIEF SUMMARY OF THE INVENTION

Initial asexual reproduction of ‘PACtwo’ by cuttings was first done in Santa Barbara, Calif., USA. The reproduction was conducted in controlled greenhouse environments.

Have here proven that the foregoing and all after characteristics and distinctions to come true to form and are established in succeeding propagations. ‘PACtwo’ is a low and compact miniature rose with good vigor. The shelflife for the entire plant and the single flush, has been tested to be good.

The objective of the hybridization of this rose variety for commercial greenhouse culture was to create a new and distinct variety with:
1. Uniform and abundant flowers with good keepability;
2. Attractive long lasting foliage and compact growth,
3. Year round flowering under glasshouse conditions;
4. Suitability for production from softwood cuttings in pots;
5. Durable flowers and foliage which make the variety suitable for distribution in the floral industry.

This combination of qualities was not present in previously available commercial cultivars of this type and distinguish ‘PACtwo’ from all other varieties of which we are aware.

The seeds from hybridization were planted in a controlled environment and evaluations were conducted on the resulting plants. ‘PACtwo’ was selected by, Dan Jauchen, in his development program in Santa Barbara, Calif., USA.

### BRIEF DESCRIPTION OF THE DRAWINGS

The accompanying color illustrations show as true as is reasonably to obtain in color photographs of this type, the typical characteristics of the buds, flowers, leaves, stems of ‘PACtwo’. Specifically illustrated in:

### Photo sheet # 1:

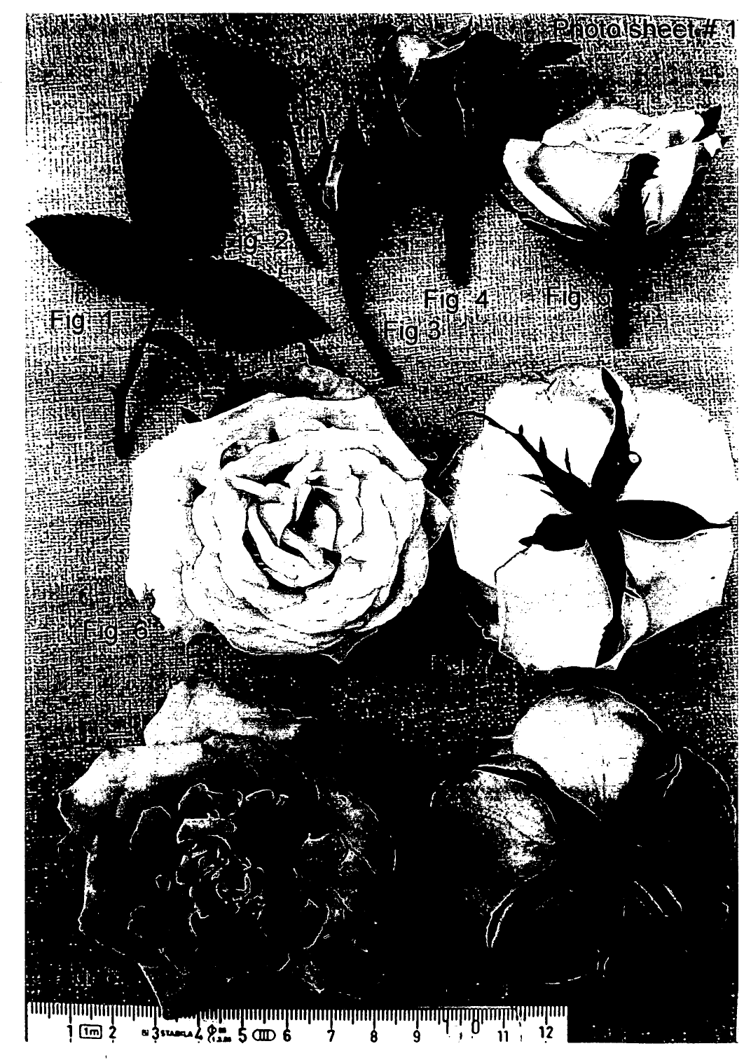

FIG. 1 Young shoot.

FIG. 2 Bud before opening the sepals.

FIG. 3 Bud at the stage of opening the sepals.

FIG. 4 Bud at the stage of opening the petals.

FIG. 5 Flower during course of opening.

FIG. 6 Open flower — plan view — obverse.

FIG. 7 Open flower — plan view — reverse.

FIG. 8 Fully open flower — plan view — obverse.

FIG. 9 Fully open flower — plan view — reverse.

### Photo Sheet # 2:

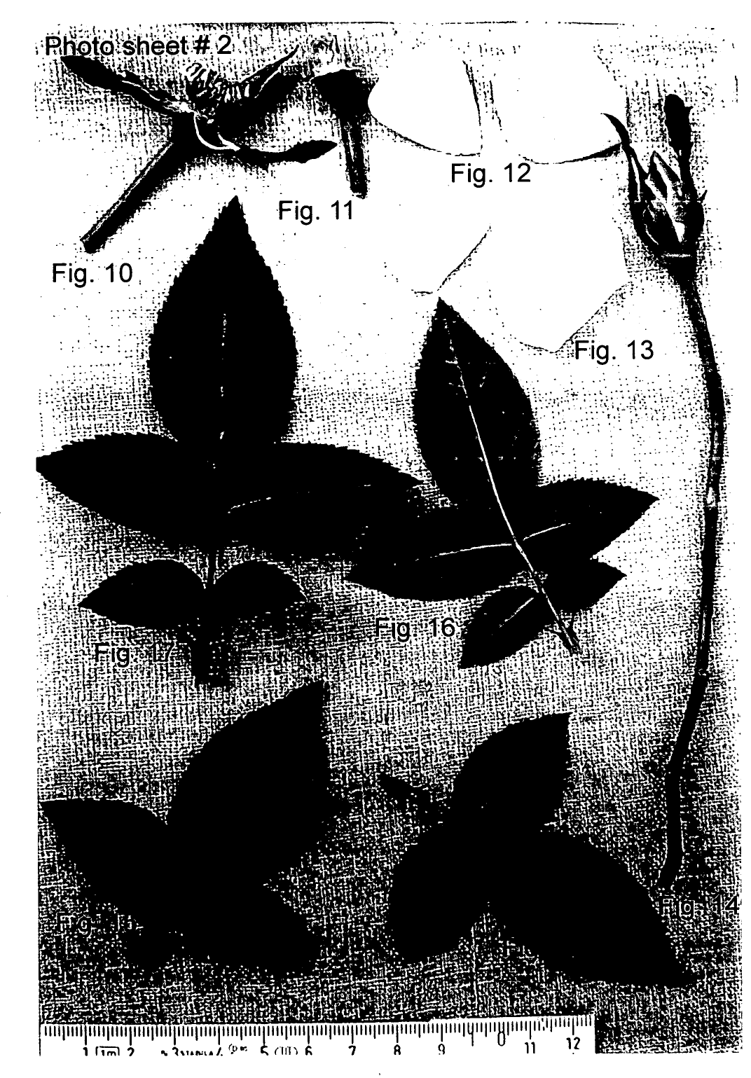

FIG. 10 Receptacle showing stamens and pistils.

FIG. 11 Receptacle showing pistils (stamens and sepals removed).

FIG. 12 Flower petals, detached — inner surface.

FIG. 13 Flower petals, detached — outer surface.

FIG. 14 Bare stem exhibiting thorns and flower attachment.

FIG. 15 Three leaflets upper side.

FIG. 16 Five leaflets reverse side.

FIG. 17 Five leaflets upper side.

FIG. 18 Three leaflets reverse side.

### DETAILED BOTANICAL DESCRIPTION OF THE VARIETY

The following is a detailed description of the Miniature Rose: Rosa hybrid ‘PACtwo’.

The following observations, measurements, values and comparisons describe plants grown in glass houses in Santa Barbara, Calif., USA.

The age of the observed plants where 11 to 13 weeks after propagation by cuttings, and produced as flowering pot plants in container of 15 centimeter in diameter.

Color references are made using The Royal Horticultural Society (London, England) Colour Chart, 1995, except where common terms of color are used. For a comparison, the nearest existing rose variety is ‘POULberty’, a rose variety described and illustrated in U.S. Plant Pat. No. PP 13,103.

Chart 1 details several physical characteristics of ‘PACtwo’ and ‘POULberty’.

CHART 1

| | ’PACtwo’ | ’POULberty’ |
|---|---|---|
| Petal color, Upper | Red Group | Yellow-Orange Group |
| surface | 49A | 20 B |
| Petal color, Reverse | Red Group | Yellow-Orange Group |
| surface | 49B | 19 B |
| Petal count | 30-35 | 25-30 |

Parents: Unnamed seedling. Times. Unnamed seedling.
Classification:
   Botanical.—Rosa hybrid.
   Commercial.—Miniature.
Plant:
   Plant growth.—Moderately vigorous. Grows compact upright to bushy. When grown as 10 cm pot plant, the average height of the plant itself is 18 to 20 cm, and average width is 20 cm. When grown as a 15 cm pot plant, the average height of the plant itself is 22 to 27 cm, and average width is 30 cm. Production time is generally 11 to 13 weeks depending on average temperature, light level, and cultural practices.
Stem:
   Color.—Young wood: Yellow-Green Group 146A. Older wood: Yellow-Green Group 148B.
   Thorns.—Incidence: Low number of thorns Size: 2 — 3 mm Color: Greyed-Purple Group 184D Shape: Deep concave.
   Surface.—Young wood: Smooth Older wood: Smooth.
   Stem diameter.—2 — 4 mm.
   Internode length.—20 — 30 mm.
   Numbers of internodes.—5-6.
Plant foliage: Leaves arranged alternately, compound with 3.5 leaflets per leaf, generally symmetrical, abundant, and flat in aspect. Stipules at petiole base.
   Quantity of leaves.—5 to 6 per lateral branch.
   Leaf size.—Medium 90 — 100 mm(l). times. 75 — 65mm(w), for 5 leaflet.
   Petioles.—Color: Yellow-Green Group 148B-C, with intonations of Greyed-Purple Group 182A Margins: with small prickles Greyed-Purple Group 182A Length: 10-15 mm Diameter: about 0.5-1 mm.
   Stipules.—Size: 10-15 mm Surface: Smooth Color: Yellow-Green Group 14BB-C, Margins: Serrated single.
   Rachis.—Color: Yellow-Green Group 148B-C, with intonations of Greyed-Purple Group 182A Margins: With small prickles, Greyed-Purple Group 182A. Length: 30 to 30 mm.
   Leaflets.—Edge: Serrated Serration: Single Shape: Ovate with acute apex and obtuse base Texture: Smooth Appearance: Dull Size: length: 30 to 35 mm. Width: 10 to 18 mm Color: Young foliage: Upper surface: Yellow-Green Group 147A With intonations of Greyed-Red 182A Lower surface: Yellow-Green Group 147C, With intonations of Greyed-Red 182A Color: Mature foliage: Upper surface: Yellow-Green Group 147A Lower surface: Greyed-Green Group 191B.
Inflorescense:
   Blooming habit.—Recurrent.
   Number of flowers.—Generally 1 bud per flowering stem.
   Peduncle.—Color: Yellow-Green Group 147C, Texture: Smooth Length: 40-50 mm Strength: Upright.
   Receptacle.—Surface: Smooth, glabrous. Shape: Funnel-shaped Size: h: 6-7 mm w: 8-10 mm Color: Yellow-Green Group 147C.
   Sepals.—Quantity: 5 Shape: Narrowly Ovate w. acute tip Texture: Leathery Margin: Foliaceous appendages on 3 of the five sepals Appearance: Dull Color: Upper surface: Yellow-Green Group 147B Reverse surface: Greyed-Green Group 190B Size: 30 mm(L), 10 mm(W).
   Buds.—Size: 18-22 mm (h) 18-20 mm (w)upon opening. Shape: Cupped Color: at % opening, Orange Group 29C.
Flower:
   Duration.—As a pot plant, flowers last from 12 to 16 days. Fragrance. None. Size: 70-75 mm in diameter. Form: Shape of flower when viewed from the side. Up on opening: Cupped to pointed Open flower: Flat Color: Petals, upon opening. Upper surface: Yellow Group 4D, With intonations of Red Group 55C on outer petals. Yellow-Orange Group 20C on inner petals. Reverse surface: Yellow Group 4D, With intonations of Red Group 55C on outer petals Yellow-Orange Group 20C on inner petals. Petals after opening: Upper surface: Yellow-Orange Group 19D , With intonations of Red Group 55C on outer petals. Yellow-Orange Group 19D on inner petals. Reverse surface: Yellow-Orange Group 19D, With intonations of Red Group 55C on outer petals General tonality: On Open flower: Third day: Orange Group 27A Afterwards: Yellow-Orange Group 19D With intonations of Red Group 55C.
Petals:
   Petal reflex.—Outermost petals reflex backwards at opening . Fully open all petals reflex backwards.
   Texture.—Smooth.
   Petal edge.—Entire.
   Petal count.—Approximately 30 on the average per flower.
   Petal size.—Length 30-35 mm Width: 25-35 mm.
   Shape.—Outer petals: Round — ovate Inner petals: Ovate Apex: Orbicular, Base: Obtuse to rotundate.
Reproductive organs:
   Stamen number.—Approximately 50 on average per flower.
   Pollen.—Color: Yellow-Orange Group 19D, Abundance: high.
   Anthers.—Size: 2-3 mm Color: Greyed-Orange Group 165D, Shape: Oblong.
   Filaments.—Size: 3-5 mm Color: Greyed-Purple Group 186D.
   Pistils number.—Approximately 20-25 on average per flower.
   Stigmas.—Location: Lower in location to anthers Color: Orange-White Group 159B.
   Styles.—Color: White Group 155B. Length: 1 to 2 mm.
Development:
   Vegetation.—Dense.
   Blooming.—Abundant.
   Aptitude to bear fruit.—Poor.
   Resistance to diseases.—Above average resistance to mildew and Botrytis under normal growing conditions in Santa Barbara, Calif., USA. Hips/seeds has not been observed due to that the plant has never been grown to the stage of seed development, due to the fact, that the variety is developed for use as a flowering pot plant only.
Winter hardiness & drought/heat tolerance: Due to the fact, that this variety is a potted flowering plant, developed indoor use only, the plant is not tested for winter hardiness or drought/heat tolerance.

1. A new and distinct variety of rose plant of the miniature class, substantially as herein illustrated and described as a distinct and novel rose variety due to its abundant yellow flowers, attractive long lasting foliage, vigorous and compact growth, year round flowering under glasshouse conditions, suitability for production from softwood cuttings in pots, and durable flowers and foliage which make the variety suitable for distribution in the floral industry.
